(19) United States
(12) Patent Application Publication
(10) Pub. No.: US 2022/0412759 A1
(43) Pub. Date: Dec. 29, 2022

(54) Navigation Prediction Vehicle Assistant

(71) Applicant: Ford Global Technologies, LLC, Dearborn, MI (US)

(72) Inventors: Cynthia Neubecker, Westland, MI (US); Brian Bennie, Sterling Heights, MI (US)

(73) Assignee: Ford Global Technologies, LLC, Dearborn, MI (US)

(21) Appl. No.: 17/358,969

(22) Filed: Jun. 25, 2021

(51) Int. Cl. G01C 21/34; G01C 21/00; G01C 21/36; A61B 5/117; A61B 5/18; A61B 5/16; A61B 5/0205; A61B 5/024; A61B 5/08; A61B 5/021

(52) U.S. Cl. CPC G01C 21/3484; G01C 21/3807; G01C 21/365; G01C 21/3629; G01C 21/3661; A61B 5/117; A61B 5/18; A61B 5/163; A61B 5/165; A61B 5/0205; A61B 5/024; A61B 5/0816; A61B 5/021; A61B 2503/22

(57) ABSTRACT

A method for controlling a vehicle can include determining a driver state based on physiological response of an operator and navigational irregularities from observed driving patterns. The physiological response may indicate observed driver stress based on bodily responses that can include respiration, heart rate, ocular movement, or other stress indicators. The method further includes determining a vehicle route having a trip start position, a path to a present position, and a trip destination, and identifying a navigation irregularity based on the vehicle route, the driver state, and a historic record driving patterns. The method may include displaying a navigation assistant output on a heads-up Human Machine Interface (HMI) based on the navigation irregularity and the physiological response of the user. The system may provide user-selectable navigation assistance including placing a phone call to a family member for navigation guidance, providing turn-by-turn navigation guidance via the heads-up HMI, and/or other measures.

### TECHNICAL FIELD

The present disclosure relates to vehicle navigational assist systems, and more particularly, to a navigation prediction vehicle assistant configured to detect biological and navigational cues to provide predicted useful driver assist features.

### BACKGROUND

Sometimes people may get lost in familiar places while driving. The reasons for losing their way while on the road can be quite diverse, including health limitations or inexperience. Additionally, as vehicles and smart devices routinely include global positioning systems (GPS), drivers may become reliant on the navigational features and may not improve their self-navigation skills over time.

When these unfamiliar situations are encountered by inexperienced or health-challenged vehicle operators, their anxiety levels may increase as their confusion and uncertainty increases. Certain driving challenges such as construction areas and detours may affect the vehicle operator negatively. Some vehicle operators may find it embarrassing or uncomfortable to ask others for help along the way. When experienced help is not readily available to provide assistance, family members may worry about safe arrival of their loved ones on the road.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example computing environment in which techniques and structures for providing the systems and methods disclosed herein may be implemented.

FIG. 2 depicts an example Navigation Prediction Vehicle Assistant Engine for providing augmented reality navigation guidance in accordance with the present disclosure.

FIG. 3 depicts an example navigation prediction controller integrated with vehicle systems in accordance with the present disclosure.

FIG. 4 depicts a flow diagram of an example method for controlling a vehicle in accordance with the present disclosure.

### DETAILED DESCRIPTION

### Overview

The systems and methods disclosed herein are configured and/or programmed to provide intelligent navigation prediction vehicle assistance. The disclosed system utilizes vehicle sensing technology, vehicle computing and data storage, and portable personal profile information (when such data is available) to monitor and predict if a driver is lost, and recommend actions to mitigate the issue.

The disclosed navigation prediction vehicle assistant may utilize vehicle interior sensory technologies (for example, cameras, radars, sounds, WIFI, phone-as-a-key (PAAK), etc.) and vehicle computing systems to determine (i) an identity of vehicle occupants, (ii) irregularities in vehicle route and operation by comparing present trip metrics and route data with historical records associated with the user operating the vehicle, and (iii) the operator's stress level and/or emotions by observing physiological responses during vehicle operation.

For example, in some embodiments, the navigation prediction vehicle assistant may monitor the physiological state of the driver using the interior sensory technologies, and use that information with route and navigational irregularity data to determine a level of relative distress associated with displacing the planned route. The navigation prediction vehicle assistant may optionally perform navigational assistance steps that can include providing step-by-step guidance using the heads-up display (HUD) or other interface output, place a call to a family member or trusted contact, or provide navigation to previously traveled locations on the present trip to obtain forgotten items or for other reasons.

One or more embodiments may include monitoring a vehicle route. For example, the assistant system may track turn-by-turn vehicle maneuvers using information from the ignition cycle, steering wheel and/or drive axle movement data, and global positioning system (GPS) record. The system may further create breadcrumb map, and compare the breadcrumb map data with overtime data to predict irregular deviations from a predicted or planned path. The overtime data may be and/or include a historic database of driving patterns and/or paths associated with the user such that the system may evaluate data associated with the present trip (e.g., turns with respect to a known destination, acceleration information, braking information, stops, indicators of navigational hesitation, indications of navigational confusion (e.g., the driver is lost), and/or other aspects of present trip data.

The assistant system may include a third function for predicting irregularities. The vehicle may automatically compare the overtime data with current data and detects unusual driving maneuvers as well increases in driver stress levels. For example, overtime data indicative of a propensity for missing turns, driving slowly at intersections, last-second lane changes near intersections followed by a turn, etc., may be compared with present driving information associated with a current trip (e.g., the breadcrumb map) having vehicle metrics and navigation metric. If the system determines that the identified user has a historic tendency to miss turns and/or perform erratic maneuvers, the system may determine a relative low probability that the user is currently lost. This determination may be heavily weighted based on the user state, which may or may not show a relative level of user stress (e.g., heavy respiration, rapid heartbeat, shifting eye movement indicating that the driver is confused about present location or a next logical navigation step, etc.).

Using a fourth interface function, the assistant system may generate an augmented reality, audio, visual, or other output using a human-machine interface (HMI). The HMI may be, in some embodiments, a Heads-Up Display (HUD) HMI. The output may suggest user-initiation of a lost mode. In the lost mode, the HUD HMI may display a help icon with an interactive question and answer (Q&A). For example, the assistant may ask if the driver needs assistance reaching a destination, would desire a phone call initiated to a relative, or may show the breadcrumbs map even if the vehicle has lost GPS connectivity.

Aspects of the present disclosure may provide navigation assistance that is customized to a unique vehicle operator utilizing facial recognition and/or other biometric identification techniques, and personalize lost mode assist features based on stored use cases and unique information unique to the particular user. The system may provide a specialized display with a driver coach to assist the user's navigation, and continuously monitor calibrations based on current driver metrics such as driver age, driver identity, driving experience, time of day, and other information. The navigation prediction vehicle assistant system 107 may provide confidence and stress reduction for new drivers, aged drivers, and others that may experience physiological stress associated with navigational confusion that is commensurate with physiological responses observed in the driver. Moreover, the disclosed system may provide interactive features that change based on dynamically changing environmental factors such as time of day, traffic or accidents in the area, and other similar factors.

These and other advantages of the present disclosure are provided in greater detail herein.

### Illustrative Embodiments

The disclosure will be described more fully hereinafter with reference to the accompanying drawings, in which example embodiments of the disclosure are shown, and not intended to be limiting.

The disclosure will be described more fully hereinafter with reference to the accompanying drawings, in which example embodiments of the disclosure are shown, and not intended to be limiting.

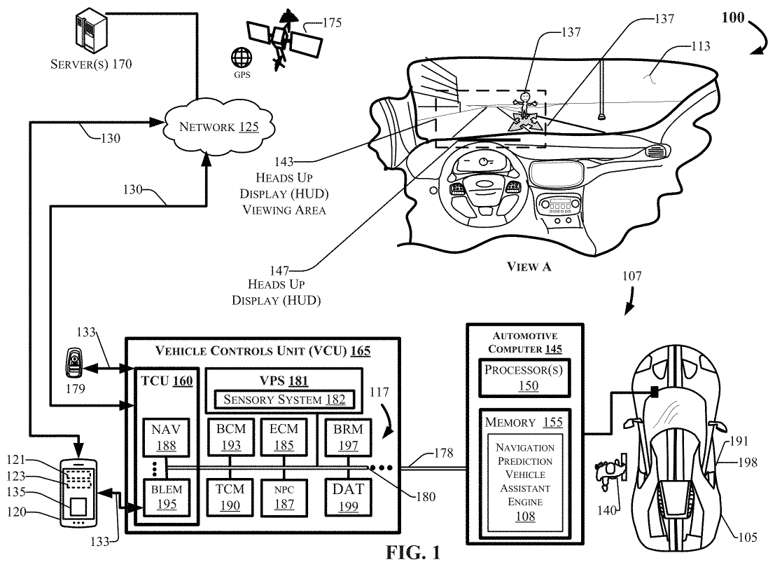

FIG. 1 depicts an example computing environment 100 that can include a vehicle 105 comprising an automotive computer 145, and a Vehicle Controls Unit (VCU) 165 that typically includes a plurality of electronic control units (ECUs) 117 disposed in communication with the automotive computer 145. A mobile device 120, which may be associated with a user 140 and the vehicle 105, may connect with the automotive computer 145 using wired and/or wireless communication protocols and transceivers to perform aspects of the present disclosure such as interfacing with a wearable smart device (not shown in FIG. 1) that obtains and/or measures physiological responses such as heart rate, temperature, respiration, eye movement, or other stress-indicating factors. The mobile device 120 may also receive instructions from a navigation prediction vehicle assistant system 107 to place a telephonic call to one or more trusted contacts of the user 140 when the system 107 determines that the user 140 may be lost or experiencing stress due to navigational difficulty.

The mobile device 120 may be communicatively coupled with the vehicle 105 via one or more network(s) 125, which may communicate via one or more wireless connection(s) 130, and/or may connect with the vehicle 105 directly using near field communication (NFC) protocols, Bluetooth® protocols, Wi-Fi, Ultra-Wide Band (UWB), and other possible data connection and sharing techniques.

The vehicle 105 may also receive and/or be in communication with a Global Positioning System (GPS) 175. The GPS 175 may be a satellite system (as depicted in FIG. 1) such as the global navigation satellite system (GLNSS), Galileo, or navigation or other similar system. In other aspects, the GPS 175 may be a terrestrial-based navigation network, or any other type of positioning technology known in the art of wireless navigation assistance. The navigation prediction vehicle assistant system 107 may utilize the GPS 175 to receive GPS location information, and utilize the GPS location information in part to identify a navigation irregularity based on the vehicle route. For example, the system 107 may track the vehicle position and maneuvers over time (e.g., generate overtime data), and generate the overtime data, which can include historic driving patterns associated with the user. The system 107 may update a breadcrumb database (not shown in FIG. 1) stored in a computer memory 155 and/or stored in one or more remote server(s) (e.g., the server(s) 170). The breadcrumb database may include a plurality of historic navigation maneuvers having turn-by-turn navigation data associated with a current trip. The system 107 may further utilize the breadcrumb database to generate and/or compile a breadcrumb map of the current trip based on the breadcrumb database.

The automotive computer 145 may be or include an electronic vehicle controller, having one or more processor(s) 150 and the memory 155. The automotive computer 145 may, in some example embodiments, be disposed in communication with the mobile device 120, and one or more server(s) 170. The server(s) 170 may be part of a cloud-based computing infrastructure, and may be associated with and/or include a Telematics Service Delivery Network (SDN) that provides digital data services to the vehicle 105 and other vehicles (not shown in FIG. 1) that may be part of a vehicle fleet. The memory 155 may store program code that can include the navigation prediction vehicle assistant engine 108, and/or may store a breadcrumb database, compiled/generated breadcrumb maps, user profile information, user overtime data, etc. (not shown in FIG. 1).

Although illustrated as a performance vehicle, the vehicle 105 may take the form of another passenger or commercial automobile such as, for example, a car, a truck, a sport utility, a crossover vehicle, a van, a minivan, a taxi, a bus, etc., and may be configured and/or programmed to include various types of automotive drive systems. Example drive systems can include various types of internal combustion engine (ICE) powertrains having a gasoline, diesel, or natural gas-powered combustion engine with conventional drive components such as, a transmission, a drive shaft, a differential, etc. In another configuration, the vehicle 105 may be configured as an electric vehicle (EV). More particularly, the vehicle 105 may include a battery EV (BEV) drive system, or be configured as a hybrid EV (HEV) having an independent onboard powerplant, a plug-in HEV (PHEV) that includes a HEV powertrain connectable to an external power source, and/or includes a parallel or series hybrid powertrain having a combustion engine powerplant and one or more EV drive systems. HEVs may further include battery and/or supercapacitor banks for power storage, flywheel power storage systems, or other power generation and storage infrastructure. The vehicle 105 may be further configured as a fuel cell vehicle (FCV) that converts liquid or solid fuel to usable power using a fuel cell, (e.g., a hydrogen fuel cell vehicle (HFCV) powertrain, etc.) and/or any combination of these drive systems and components.

Further, the vehicle 105 may be a manually driven vehicle, and/or be configured and/or programmed to operate in one or more partial autonomy modes. Examples of partial autonomy modes are widely understood in the art as autonomy Levels 0 through 4. A vehicle having a Level-0 autonomous automation may not include autonomous driving features. An autonomous vehicle (AV) having Level-1 autonomy may generally include a single automated driver assistance feature, such as steering or acceleration assistance. Adaptive cruise control is one such example of a Level-1 autonomous system that includes aspects of both acceleration and steering. Level-2 autonomy in vehicles may provide partial automation of steering and acceleration functionality, where the automated system(s) are supervised by a human driver that performs non-automated operations such as braking and other controls. Level-3 autonomy in a vehicle can generally provide conditional automation and control of driving features. For example, Level-3 vehicle autonomy typically includes “environmental detection” capabilities, where the vehicle can make informed decisions independently from a present driver, such as accelerating past a slow-moving vehicle, while the present driver remains ready to retake control of the vehicle if the system is unable to execute the task. Level-4 autonomy includes vehicles having high levels of autonomy that can operate independently from a human driver, but still include human controls for override operation. Level-4 automation may also enable a self-driving mode to intervene responsive to a predefined conditional trigger, such as a road hazard or a system failure. Accordingly, the navigation prediction vehicle assistant system 107 may provide some aspects of human control to the vehicle 105, when the vehicle is configured with driver assistances technologies. Accordingly, the system 107 may work in conjunction with automated driving features in instances where the vehicle control is performed at least in part by the user 140.

The mobile device 120 generally includes a memory 123 for storing program instructions associated with an application 135 that, when executed by a mobile device processor 121, performs aspects of the disclosed embodiments. The application (or “app”) 135 may be part of the navigation prediction vehicle assistant system 107, or may provide information to the navigation prediction vehicle assistant system 107 and/or receive information from the navigation prediction vehicle assistant system 107. For example, the app 135 may provide user prompts such as an output asking the user 140 whether they are lost and would like to engage the navigation prediction vehicle assistant guidance system, place a phone call to a family member, re-trace their navigation pathway to proceed to a site visited along the trip route (e.g., along the breadcrumb data map), and/or provide other communication prompts and receive user communication responses.

In some aspects, the mobile device 120 may communicate with the vehicle 105 through the one or more wireless connection(s) 130, which may be encrypted and established between the mobile device 120 and a Telematics Control Unit (TCU) 160. The mobile device 120 may communicate with the TCU 160 using a wireless transmitter (not shown in FIG. 1) associated with the TCU 160 on the vehicle 105. The transmitter may communicate with the mobile device 120 using a wireless communication network such as, for example, the one or more network(s) 125. The wireless connection(s) 130 are depicted in FIG. 1 as communicating via the one or more network(s) 125, and via one or more wireless connection(s) 133 that can be direct connection(s) between the vehicle 105 and the mobile device 120 and/or the fob 179. The wireless connection(s) 133 may include various low-energy protocols including, for example, Bluetooth®, BLE, or other Near Field Communication (NFC) protocols.

The network(s) 125 illustrate an example communication infrastructure in which the connected devices discussed in various embodiments of this disclosure may communicate. The network(s) 125 may be and/or include the Internet, a private network, public network or other configuration that operates using any one or more known communication protocols such as, for example, transmission control protocol/Internet protocol (TCP/IP), Bluetooth®, Wi-Fi based on the Institute of Electrical and Electronics Engineers (IEEE) standard 802.11, Ultra-Wide Band (UWB), and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few examples.

The automotive computer 145 may be installed in an engine compartment of the vehicle 105 (or elsewhere in the vehicle 105) and operate as a functional part of the navigation prediction vehicle assistant system 107, in accordance with the disclosure. The automotive computer 145 may include one or more processor(s) 150 and a computer-readable memory 155.

The one or more processor(s) 150 may be disposed in communication with one or more memory devices disposed in communication with the respective computing systems (e.g., the memory 155 and/or one or more external databases not shown in FIG. 1). The processor(s) 150 may utilize the memory 155 to store programs in code and/or to store data for performing aspects in accordance with the disclosure. The memory 155 may be a non-transitory computer-readable memory storing an enhanced pathway program code. The memory 155 can include any one or a combination of volatile memory elements (e.g., dynamic random access memory (DRAM), synchronous dynamic random access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.

The VCU 165 may share a power bus 178 with the automotive computer 145, and may be configured and/or programmed to coordinate the data between vehicle 105 systems, connected servers (e.g., the server(s) 170), and other vehicles (not shown in FIG. 1) operating as part of a vehicle fleet. The VCU 165 can include or communicate with any combination of the ECUs 117, such as, for example, a Body Control Module (BCM) 193, an Engine Control Module (ECM) 185, a Transmission Control Module (TCM) 190, the TCU 160, a Navigation Prediction Controller (NPC) 187, a Driver Assistances Technologies (DAT) controller 199, etc. The VCU 165 may further include and/or communicate with a Vehicle Perception System (VPS) 181, having connectivity with and/or control of one or more vehicle sensory system(s) 182. In some aspects, the VCU 165 may control operational aspects of the vehicle 105, and implement one or more instruction sets received from the application 135 operating on the mobile device 120, from one or more instruction sets stored in computer memory 155 of the automotive computer 145, including instructions operational as part of the navigation prediction vehicle assistant system 107.

The TCU 160 can be configured and/or programmed to provide vehicle connectivity to wireless computing systems onboard and offboard the vehicle 105, and may include a Navigation (NAV) receiver 188 for receiving and processing a GPS signal from the GPS 175, a Bluetooth® Low-Energy (BLE) Module (BLEM) 195, a Wi-Fi transceiver, an Ultra-Wide Band (UWB) transceiver, and/or other wireless transceivers (not shown in FIG. 1) that may be configurable for wireless communication between the vehicle 105 and other systems, computers, and modules. The TCU 160 may be disposed in communication with the ECUs 117 by way of a bus 180. In some aspects, the TCU 160 may retrieve data and send data as a node in a CAN bus.

The BLEM 195 may establish wireless communication using Bluetooth® and Bluetooth Low-Energy® communication protocols by broadcasting and/or listening for broadcasts of small advertising packets, and establishing connections with responsive devices that are configured according to embodiments described herein. For example, the BLEM 195 may include Generic Attribute Profile (GATT) device connectivity for client devices that respond to or initiate GATT commands and requests, and connect directly with the mobile device 120.

The bus 180 may be configured as a Controller Area Network (CAN) bus organized with a multi-master serial bus standard for connecting two or more of the ECUs 117 as nodes using a message-based protocol that can be configured and/or programmed to allow the ECUs 117 to communicate with each other. The bus 180 may be or include a high speed CAN (which may have bit speeds up to 1 Mb/s on CAN, 5 Mb/s on CAN Flexible Data Rate (CAN FD)), and can include a low-speed or fault tolerant CAN (up to 125 Kbps), which may, in some configurations, use a linear bus configuration. In some aspects, the ECUs 117 may communicate with a host computer (e.g., the automotive computer 145, the navigation prediction vehicle assistant system 107, and/or the server(s) 170, etc.), and may also communicate with one another without the necessity of a host computer. The bus 180 may connect the ECUs 117 with the automotive computer 145 such that the automotive computer 145 may retrieve information from, send information to, and otherwise interact with the ECUs 117 to perform steps described according to embodiments of the present disclosure. The bus 180 may connect CAN bus nodes (e.g., the ECUs 117) to each other through a two-wire bus, which may be a twisted pair having a nominal characteristic impedance. The bus 180 may also be accomplished using other communication protocol solutions, such as Media Oriented Systems Transport (MOST) or Ethernet. In other aspects, the bus 180 may be a wireless intra-vehicle bus.

The VCU 165 may control various loads directly via the bus 180 communication or implement such control in conjunction with the BCM 193. The ECUs 117 described with respect to the VCU 165 are provided for example purposes only, and are not intended to be limiting or exclusive. Control and/or communication with other control modules not shown in FIG. 1 is possible, and such control is contemplated.

In an example embodiment, the ECUs 117 may control aspects of vehicle operation and communication using inputs from human drivers, inputs from an autonomous vehicle controller, the navigation prediction vehicle assistant system 107, and/or via wireless signal inputs received via the wireless connection(s) 133 from other connected devices such as the mobile device 120 and the fob 179, among others. The ECUs 117, when configured as nodes in the bus 180, may each include a central processing unit (CPU), a CAN controller, and/or a transceiver (not shown in FIG. 1). For example, although the mobile device 120 is depicted in FIG. 1 as connecting to the vehicle 105 via the BLEM 195, it is possible and contemplated that the wireless connection 133 may also or alternatively be established between the mobile device 120 and one or more of the ECUs 117 via the respective transceiver(s) associated with the module(s).

The BCM 193 generally includes integration of sensors, vehicle performance indicators, and variable reactors associated with vehicle systems, and may include processor-based power distribution circuitry that can control functions associated with the vehicle body such as lights, windows, security, door locks and access control, and various comfort controls. The BCM 193 may also operate as a gateway for bus and network interfaces to interact with remote ECUs (not shown in FIG. 1).

The BCM 193 may coordinate any one or more functions from a wide range of vehicle functionality, including energy management systems, alarms, vehicle immobilizers, driver and rider access authorization systems, Phone-as-a-Key (PaaK) systems, driver assistance systems, AV control systems, power windows, doors, actuators, and other functionality, etc. The BCM 193 may be configured for vehicle energy management, exterior lighting control, wiper functionality, power window and door functionality, heating ventilation and air conditioning systems, and driver integration systems. In other aspects, the BCM 193 may control auxiliary equipment functionality, and/or be responsible for integration of such functionality.

In some aspects, the vehicle 105 may include one or more Door Access Panels (DAPs) 191 disposed on exterior door surface(s) of vehicle door(s) 198, and connected with a DAP controller (not shown in FIG. 1). In some aspects, the user 140 may have the option of entering a vehicle by typing in a personal identification number (PIN) on an exterior interface associated with a vehicle. The user interface may be included as part of a Door Access Panel (DAP) 191, a wireless keypad, included as a part of the mobile device 120, or be included as part of another interface. The DAP 191, which may operate and/or communicate with the NPC 187 or another of the ECUs 117, can include and/or connect with an interface with which the user 140 may input identification credentials and receive information from the system. Accordingly, the navigation prediction vehicle assistant system 107 may connect with the DAP 191 and receive vehicle access credentials that uniquely identify the user 140 such that the navigation prediction vehicle assistant engine 108 may utilize stored information associated with the user's unique identity such as age, physical and/or mental dispositions, propensity to become navigationally lost, erratic driving patterns, etc.

In one aspect, the DAP 191 may be disposed on a vehicle door 198, a vehicle pillar (not shown in FIG. 1), and/or another vehicle exterior surface, and can include an interface device (not shown in FIG. 1) from which the user 140 can interact with the system by selecting their unique identifier from a list, and by entering personal identification numbers (PINs) and other non-personally identifying information. In some embodiments, the interface may be a mobile device, a keypad, a wireless or wired input device, a vehicle infotainment system, and/or the like. Accordingly, it should be appreciated that, although a DAP is described with respect to embodiments herein, the interface may alternatively be one or more other types of interfaces described above.

The NPC 187 can include sensory and processor functionality and hardware to facilitate user and device authentication, and provide occupant customizations and support that provide customized experiences for vehicle occupants. The NPC 187 may connect with a Driver Assist Technologies (DAT) controller 199 configured and/or programmed to provide biometric authentication controls, including, for example, facial recognition, fingerprint recognition, voice recognition, and/or other information associated with characterization, identification, and/or verification for other human factors such as gait recognition, body heat signatures, eye tracking, etc.

The DAT controller 199 may also provide Level-1 through Level-3 automated driving and driver assistance functionality that can include, for example, active parking assistance, trailer backup assistance, adaptive cruise control, lane keeping, and/or driver status monitoring, among other features. The DAT controller 199 may also provide aspects of user and environmental inputs usable for user authentication. Authentication features may include, for example, biometric authentication and recognition. For example, in one example embodiment, the DAT controller 199 may determine a user identity associated with the user 140 based on user profile information (not shown in FIG. 1) that stores user 140 biometric information such as iris identification data, voice recognition data, facial feature recognition data, gate recognition data, among other identifying biometrics. The system 107 may utilize the user identity information to determine driver state metrics that may identify or indicate a probability of physiological distress associated with navigational difficulties.

The DAT controller 199 can obtain input information via the sensory system(s) 182, which may include sensors disposed on the vehicle interior and/or exterior (sensors not shown in FIG. 1). The DAT controller 199 may receive the sensor information associated with driver functions, vehicle functions, and environmental inputs, and other information. The DAT controller 199 may characterize the sensor information for identification of biometric markers stored in a secure biometric data vault (not shown in FIG. 1) onboard the vehicle 105 and/or via the server(s) 170 based on unique user identity determined at least in part using biometric identification and/or self-authentication.

With respect to biometric authentication, the vehicle 105 may determine a unique identity of the user 140 to determine whether the user 140 is a new driver, an experienced driver, a senior citizen, and/or a user with limited capacity for vehicle navigation. Among other information that may be stored and retrieved by the system 107, a driver profile (not shown in FIG. 1) associated with overtime information (e.g., a historic database of driver characteristics associated with driving patterns, habits, routes, driving speeds, etc.) may indicate a general propensity associated with a unique user (e.g., the user 140) for getting lost in navigation, for being disposed to being geographically displaced and/or confusion, etc., and/or a combination thereof. The DAT controller 199 may also include computational infrastructure for evaluating a driver (e.g., the user 140) physiological response to stimuli associated with operating the vehicle 105, such as respiration, eye movement, body temperature, perspiration, self-talk (e.g., mumbling, speaking thoughts, etc.), heart rate, blood pressure, facial expression, etc. Any one or more physiological responses to operating the vehicle, in conjunction with present trip data may be indicative that the user 140 is lost or confused while operating the vehicle 105. The DAT controller 199 may provide a quantitative value to the navigation prediction vehicle assistant engine 108 indicative of driver stress and confusion, which may cause the navigation prediction vehicle assistant engine 108 to offer navigational assistance.

In another example embodiment, the navigation prediction vehicle assistant engine may receive raw data from the VPS 181 directly and/or via the DAT, and evaluate metrics associated with driver stress and confusion. It should be appreciated that any number of architectures are contemplated, and such configurations are possible within the scope of the present disclosure.

In other aspects, the DAT controller 199 may also be configured and/or programmed to control Level-1 and/or Level-2 driver assistance when the vehicle 105 includes Level-1 or Level-2 autonomous vehicle driving features. The DAT controller 199 may connect with and/or include the Vehicle Perception System (VPS) 181, which may include internal and external sensory systems (collectively referred to as sensory systems 182). The sensory systems 182 may be configured and/or programmed to obtain sensor data usable for biometric authentication, and for performing driver assistance operations such as, for example, active parking, trailer backup assistances, adaptive cruise control and lane keeping, driver status monitoring, and/or other features.

The computing system architecture of the automotive computer 145, VCU 165, and/or the navigation prediction vehicle assistant system 107 may omit certain computing modules. It should be readily understood that the computing environment depicted in FIG. 1 is an example of a possible implementation according to the present disclosure, and thus, it should not be considered limiting or exclusive.

View A depicts a driver view (driver is not shown in View A for clarity) of a roadway through a windshield 113 configured with a Heads-Up Display (HUD) HMI 147. The HUD HMI 147 may provide animated representations of navigational instructions or steps, provide alerts or instructions in the form of readable text, icons, animations, or other output. The HUD HMI 147 may further include audio output (not shown in FIG. 1) such that instructions and alerts are also output in audio.

In some aspects, the HUD HMI 147 projects animations in a viewing area 143. The navigation prediction vehicle assistant system 107 may determine whether the user 140 is likely experiencing discomfort while driving due to navigational difficulty. The navigation prediction vehicle assistant system 107 makes this determination using various sources of data that can include a perceived indication of physiological distress (e.g., the user 140 is undergoing distress associated with operating the vehicle 105) by monitoring breathing patterns, temperature, eye movement, verbal utterings, and/or other indicators using one or more sensory system 182 devices operative as part of the VPS 181.

Responsive to identifying a navigation irregularity based on the vehicle route and the driver state indicative of physiological distress, the system 107 may display on the HUD HMI 147 an animation or other output, and provide navigation assistance to the user 140. For example, navigation assistance can include placing a phone call to a trusted contact (e.g., family member) of the user 140, providing step-by-step driving instructions to a route destination, or other similar assisting actions.

The system 107 may determine driver state using the VPS 181, which can include cabin-facing red-green-blue (RGB) and/or infrared camera sensors that track eye movement, body posture and movements, and eye movements, microphone input devices that receive audio inputs such as utterances or expressions of frustration made by the user 140, sonar devices, RADAR devices that determine body movements, posture, and position, transducer devices (e.g., one or more transducers disposed in a steering wheel that determine an abnormally tight steering wheel grip as compared to prior grip forces applied by the user 140), and/or other sensory devices known in the art. In other aspects, the system 107 may further determine driver state using physiological indicators of stress such as blood pressure, heart rate, body temperature, respiration frequency, and/or other as observed/sensed physiological responses using one or more wearable devices (not shown) such as a smart watch.

Responsive to determining that the user has difficulty in navigation (e.g., by determining the navigation irregularity), the system 107 may display the navigation assistant output on the HUD viewing area 143 using the HUD HMI 147 and generate one or more of an auditory and a visual user prompt that requests user feedback indicative of a desire to receive driving assistance. For example, the system 107 may generate an audio output alone or in conjunction with a virtual driving coach asking, “you appear to be having difficulty finding your way. Would you like me to help you?” The system 107 may receive a verbal response from the user 140, and provide navigation assistance to the user 140. The navigation assistance may be, for example, a telephonic communication to a family member offboard the vehicle 105 (family member not shown in FIG. 1) using the mobile device 120 or another connected telephonic device. In other aspects, the system 107 may output on the HUD HMI 147 a breadcrumb map (not shown in FIG. 1) that provides step-by-step instructions to reach the desired destination.

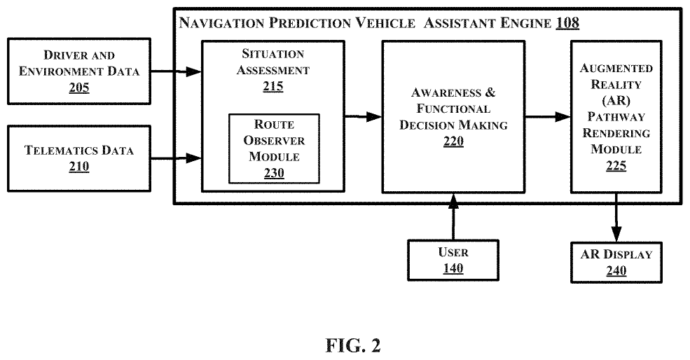

FIG. 2 depicts an example functional schematic of the navigation prediction vehicle assistant engine 108 (hereafter “NAV prediction engine 108”), which may be configured and/or programmed for providing augmented reality pathway guidance in accordance with the present disclosure. navigation prediction vehicle assistant engine 108 may receive data inputs from the VCU 165 that can include telematics data 210 and driver, route, and environment data 205.

As a general overview, the navigation prediction vehicle assistant engine 108 may include a situation assessment module 215 that delivers route observation data and other situational observations to an awareness and functional decision making module 220 that may determine appropriate navigational guidance for output via an augmented reality (AR) pathway rendering module 225 via an AR display 240.

In one example embodiment, the VCU 165 (as shown in FIG. 1) may obtain and communicate the telematics data 210 that may include vehicle operation information such as, for example, vehicle turn actions, acceleration and deceleration actions, vehicle speed, and other telematic information. The BCM 193 (shown in FIG. 1) may obtain sensory data indicative of the telematic information that may be utilized by navigation prediction vehicle assistant engine 108 to evaluate driver state and navigation irregularity.

In other aspects, navigation prediction vehicle assistant engine 108 may further receive from the VCU environmental data such as driving conditions (e.g., weather), and time of day information which may be utilized by navigation prediction vehicle assistant engine 108 to weight factors that may have a higher or lower relative likelihood of causing driver stress.

More specifically, a situation assessment module 215 may receive such inputs to assess driving and driver situations that may provide indicators of driver stress and navigational distress. The situation assessment module 215 may weight the inputs according to dynamically changing factors such as time of day. For example, navigation prediction vehicle assistant engine 108 may weight an overall predictor of driver stress based on a nighttime driving scenario when the user 140 is not normally accustomed to nighttime driving conditions. Accordingly, navigation prediction vehicle assistant engine 108 may utilize the environmental data alone or in conjunction with other historic and user-specific driver information such as user profile information and overtime data to determine driver state, and assess the dynamically changing driving environment based on the environment data 205 and telematics data 210.

In one aspect, the navigation prediction vehicle assistant engine 108 can make functional decisions for AR output based on the data inputs (e.g., the driver, route and environment data 205 and/or the telematics data 210), where the functional decisions may direct augmented reality pathway rendering on an AR display. For example, the system 107 may receive trip route data that indicates that the driver has deviated from a predicted route to the destination by a predetermined margin of error (e.g., deviating off course by 2 miles, 1 mile, six blocks, etc.), and may further receive environmental data indicative that it is night time and driving conditions are less than ideal with rain. navigation prediction vehicle assistant engine 108 may further receive sensory data from the sensory system 182 indicative that the driver is experiencing physical manifestations of stress due to being lost or for other reasons. The navigation prediction vehicle assistant engine 108 may then make one or more functional decisions that can include, for example, the driver is likely lost and may benefit from some navigational assistance or coaching from a family member, and/or As earlier explained, in one embodiment, the AR display may be a heads-up display (HUD) that provides a clear view of the vehicle pathway, upon which the navigation prediction vehicle assistant engine 108 overlays an augmented reality coach or navigation assistant 137. In another embodiment, the navigation assistant 137 may be displayed by another AR device such as a pair of operatively connected smart glasses (not shown in FIG. 1) that may be worn by the user 140.

The situation assessment module 215 may provide situation assessment using a route observer module 230. The route observer module 230 may receive driver and environment data 205 from the sensory system(s) 182, from the server(s) 170, and other online resources available via the network(s) 125. The driver and environment data may include environmental factors exterior to the vehicle 105, environmental information interior to the vehicle, and driver inputs in the form of sensory information from the sensory system(s) 182. The situation assessment module 215 may utilize the telematics data (which may further include GPS data), to generate overtime data comprising historic driving patterns associated with the user 140, update a breadcrumb database comprising a plurality of historic navigation maneuvers having turn-by-turn navigation data associated with a current trip, and compile a breadcrumb map of the current trip based on the breadcrumb database.

The awareness and functional decision making module 220 may include one or more machine learning algorithms trained to determine possible indicators of driver stress as discussed in previous portions of the present disclosure. Responsive to determining that the user 140 may benefit from guidance assistance, the awareness and functional decision making module 220 may cause the 225 to generate output on the AR display 240. The AR display 240 may be, for example, the HUD HMI 147 or another augmented reality output device (not shown in FIG. 2).

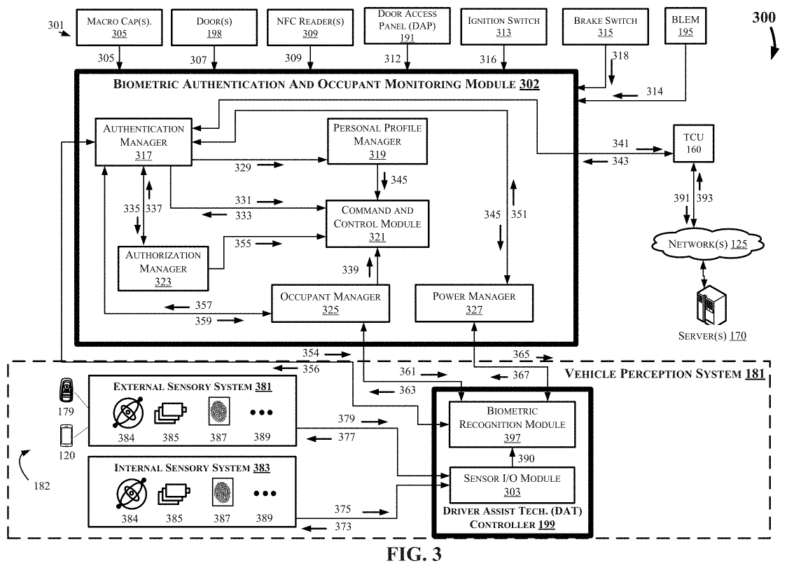

FIG. 3 illustrates a functional schematic of an example architecture of a biometric authentication and occupant monitoring system 300 that may be used for providing vehicle entry and signal authentication using biometric information and other human factors, and for providing user support and customization for the vehicle 105, in accordance with the present disclosure.

The biometric authentication and occupant monitoring system 300 may authenticate passive device signals from a Passive Entry Passive Start (PEPS)-configured device such as the mobile device 120, a passive key device such as the fob 179, and provide vehicle entry and signal authentication using biometric information and other human factors. The biometric and occupant monitoring system 300 may also provide user support and customizations to enhance user experience with the vehicle 105. The authentication and occupant monitoring system 300 can include the NPC 187, which may be disposed in communication with the DAT controller 199, the TCU 160, the BLEM 195, and a plurality of other vehicle controllers 301, which may include vehicle sensors, input devices, and mechanisms. Examples of the plurality of other vehicle controllers 301 can include, one or more macro capacitor(s) 305 that may send vehicle wakeup data 306, the door handle(s) 196 that may send PEPS wakeup data 307, near-field communication (NFC) reader(s) 309 that send NFC wakeup data 310, the DAPs 191 that send DAP wakeup data 312, an ignition switch 313 that can send a ignition switch actuation signal 316, and/or a brake switch 315 that may send a brake switch confirmation signal 318, among other possible components. The PEPS wakeup data 307 may therefore be used to uniquely identify the user 140.

The DAT controller 199 may include and/or connect with a biometric recognition module 397 disposed in communication with the DAT controller 199 via a sensor Input/Output (I/O) module 303. The NPC 187 may connect with the DAT controller 199 to provide biometric authentication controls, including, for example, facial recognition, fingerprint recognition, voice recognition, and/or other information associated with characterization, identification, and/or verification for other human factors such as gait recognition, body heat signatures, eye tracking, etc.

The DAT controller 199 may be configured and/or programmed to provide biometric authentication control for the vehicle 105, including, for example, facial recognition, fingerprint recognition, voice recognition, and/or other provide authenticating information associated with characterization, identification, occupant appearance, occupant status, and/or verification for other human factors such as gait recognition, body heat signatures, eye tracking, etc. The DAT controller 199 may obtain the sensor information from an external sensory system 381, which may include sensors disposed on a vehicle exterior and in devices connectable with the vehicle 105 such as the mobile device 120 and/or the fob 179.

The DAT controller 199 may further connect with an internal sensory system 381, which may include any number of sensors configured in the vehicle interior (e.g., the vehicle cabin, which is not depicted in FIG. 3). The external sensory system 381 and internal sensory system 383 can connect with and/or include one or more inertial measurement units (IMUs) 384, camera sensor(s) 385, fingerprint sensor(s) 387, and/or other sensor(s) 389, and obtain biometric data usable for characterization of the sensor information for identification of biometric markers stored in a secure biometric data vault (not shown in FIG. 3) onboard the vehicle 105. The DAT controller 199 may obtain, from the internal and external sensory systems 381 and 383, sensory data 379 that can include external sensor response signal(s) and internal sensor response signal(s) 375 (collectively referred to as sensory data 390), via the sensor I/O module 303. The DAT controller 199 (and more particularly, the biometric recognition module 397) may characterize the sensory data 390, and generate occupant appearance and status information to the occupant manager 325, which may use the sensory data 390 according to described embodiments.

The internal and external sensory systems 383 and 381 may provide the sensory data 379 obtained from the external sensory system 381 and the sensory data 375 from the internal sensory system 383 responsive to an internal sensor request message 373 and an external sensor request message 377, respectively. The sensory data 379 and 375 may include information from any of the sensors 384-389, where the external sensor request message 377 and/or the internal sensor request message 373 can include the sensor modality with which the respective sensor system(s) are to obtain the sensory data.

The camera sensor(s) 385 may include thermal cameras, optical cameras, and/or a hybrid camera having optical, thermal, or other sensing capabilities. Thermal cameras may provide thermal information of objects within a frame of view of the camera(s), including, for example, a heat map figure of a subject in the camera frame. An optical camera may provide a color and/or black-and-white image data of the target(s) within the camera frame. The camera sensor(s) 385 may further include static imaging, or provide a series of sampled data (e.g., a camera feed) to the biometric recognition module 397.

The IMU(s) 384 may include a gyroscope, an accelerometer, a magnetometer, or other inertial measurement device. The fingerprint sensor(s) 387 can include any number of sensor devices configured and/or programmed to obtain fingerprint information. The fingerprint sensor(s) 387 and/or the IMU(s) 384 may also be integrated with and/or communicate with a passive key device, such as, for example, the mobile device 120 and/or the fob 179. The fingerprint sensor(s) 387 and/or the IMU(s) 384 may also (or alternatively) be disposed on a vehicle exterior space such as the engine compartment (not shown in FIG. 3), door panel (not shown in FIG. 3), etc. In other aspects, when included with the internal sensory system 383, the IMU(s) 384 may be integrated in one or more modules disposed within the vehicle cabin or on another vehicle interior surface.

The biometric authentication and occupant monitoring module 302 can include an authentication manager 317, a personal profile manager 319, a command and control module 321, an authorization manager 523, an occupant manager 325, and a power manager 327, among other control components.

The authentication manager 317 may communicate biometric key information 354 to the DAT 399. The biometric key information can include biometric mode updates indicative of a particular modality with which the internal and/or external sensory systems 383 and 381 are to obtain sensory data. The biometric key information 354 may further include an acknowledgement of communication received from the biometric recognition module 397, an authentication status update including, for example, biometric indices associated with user biometric data, secured channel information, biometric location information, and/or other information. In some aspects, the authentication manager 317 may receive biometric key administration requests 356 and other responsive messages from the biometric recognition module 397, which can include, for example, biometric mode message responses and/or other acknowledgements.

The authentication manager 317 may further connect with the TCU 160 and communicate biometric status payload information 341 to the TCU 160 indicative of the biometric authentication status of the user 140, requests for key information, profile data, and other information. The TCU 160 may send and/or forward digital key payload 391 to the server(s) 170 via the network(s) 125, and receive digital key status payload 393 from the server(s) 170 and provide responsive messages and/or commands to the authentication manager 317 that can include biometric information payload 343.

Moreover, the authentication manager 317 may be disposed in communication with the BLEM 195, and/or other the other vehicle controllers and systems 301 according to embodiments described in the present disclosure. For example, the BLEM 193 may send a PaaK wakeup message, or another initiating signal indicating that one or more components should transition from a low-power mode to a ready mode.

The authentication manager 317 may also connect with the personal profile manager 319, and the power manager 327. The personal profile manager 319 may perform data management associated with user profiles, which may be stored in the automotive computer 145 and/or stored on the server(s) 170. For example, the authentication manager 317 may send occupant seat position information 329 to the personal profile manager 319, which may include a seat position index (not shown in FIG. 3) indicative of preferred and/or assigned seating for passengers of the vehicle 105. The personal profile manager 319 may update seating indices, delete and create profiles, and perform other administrative duties associated with individualized user profile management.

The power manager 327 may receive power control commands from the authentication manager 317, where the power control commands are associated with biometric authentication device management including, for example, device wakeup causing the biometric recognition module 397 and/or the DAT controller 199 to transition from a low power (standby mode) state to a higher power (e.g., active mode) state. The power manager 327 may send power control acknowledgements 351 to the authentication manager 317 responsive to the control commands 345. For example, responsive to the power and control commands 345 received from the authentication manager 317, the power manager 327 may generate a power control signal 365 and send the power control signal to the biometric recognition module. The power control signal 365 may cause the biometric recognition module to change power states (e.g., wakeup, etc.). The biometric recognition module 397 may send a power control signal response 367 to the power manager 327 indicative of completion of the power control signal 365.

The authentication manager 317 and/or the personal profile manager 319 may further connect with the command and control module 321, which may be configured and/or programmed to manage user permission levels, and control vehicle access interface(s) (not shown in FIG. 3) for interfacing with vehicle users. The command and control module 321 may be and/or include, for example, the BCM 193 described with respect to FIG. 1. For example, the authentication manager 317 may send command and control authentication information 331 that cause the command and control module 321 to actuate one or more devices according to successful or unsuccessful authentication of a device, a signal, a user, etc. The command and control module 321 may send acknowledgements 333 and other information including, for example, vehicle lock status.

The occupant manager 325 may connect with the authentication manager 317, and communicate occupant change information 357 indicative of occupant changes in the vehicle 105 to the authentication manager 317. For example, when occupants enter and exit the vehicle 105, the occupant manager 325 may update an occupant index (not shown in FIG. 3), and transmit the occupant index as part of the occupant change information 357 to the authentication manager. The occupant manager 325 may further connect with the occupant manager 536 to update the occupant manager 325 with seat indices 359, which may include confirmation messages for seat index changes, and occupant entries and exits from the vehicle 105.

The occupant manager 325 may also receive seat indices 359 from the authentication manager 317, which may index seating arrangements, positions, preferences, and other information.

The occupant manager 325 may also connect with the command and control module 321. The command and control module 321 may receive adaptive vehicle control information 339 from the occupant manager 325, which may communicate and/or include settings for vehicle media settings, seat control information, occupant device identifiers, and other information.

The occupant manager 325 may be disposed in communication with the DAT controller 199, and may communicate biometric mode update information 361 to the biometric recognition module 397, which may include instructions and commands for utilizing particular modalities of biometric data collection from the internal sensory system 383 and/or the external sensory system 381. The occupant manager 325 may further receive occupant status update information and/or occupant appearance update information (collectively shown as information 363 in FIG. 3) from the biometric recognition module 397.

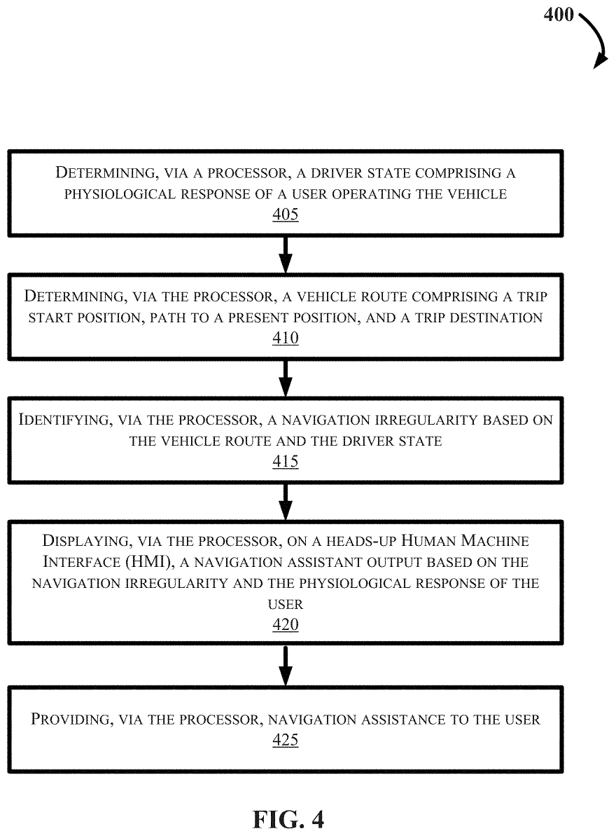

FIG. 4 is a flow diagram of an example method 400 for controlling a vehicle, according to the present disclosure. FIG. 4 may be described with continued reference to prior figures, including FIGS. 1-3. The following process is exemplary and not confined to the steps described hereafter. Moreover, alternative embodiments may include more or less steps that are shown or described herein, and may include these steps in a different order than the order described in the following example embodiments.

Referring first to FIG. 4, at step 405, the method 400 may commence with determining, via a processor, a driver state comprising a physiological response of a user operating the vehicle. This step may include determining, via the processor, a user identity based on user profile information, and determining the driver state based on the user identity. In some aspects, determining the driver state can include determining the physiological response of the user by obtaining an ocular position of the user, and determining a quantified stress level based on the ocular position of the user. The system may determine the quantified stress level in a number of ways using onboard sensory equipment and/or interfacing with wearable smart devices associated with the user. For example, determining the system may determine the physiological response of the user by sensing a user heart rate determining a quantified stress level based on the user heart rate. In other aspects, the system may determine the physiological response of the user by obtaining a user respiration frequency, and determining a quantified stress level based on the user respiration frequency. In another example embodiment, the system may determine the physiological response of the user by determining user blood pressure, and determine a quantified stress level based on the user blood pressure. Other physiological responses may be identified and quantified by the system.

At step 410, the method 400 may further include determining, via the processor, a vehicle route comprising a trip start position, path to a present position, and a trip destination.

At step 415, the method 400 may further include identifying, via the processor, a navigation irregularity based on the vehicle route and the driver state. This step may include generating overtime data comprising historic driving patterns associated with the user, updating a breadcrumb database comprising a plurality of historic navigation maneuvers having turn-by-turn navigation data associated with a current trip, and compiling a breadcrumb map of the current trip based on the breadcrumb database. This step can further include creating a breadcrumb map comparison of the breadcrumb map with the overtime data, and identifying the navigation irregularity based on the breadcrumb map comparison and the driver state.

At step 420, the method 400 may further include displaying, via the processor, on a heads-up HMI, a navigation assistant output based on the navigation irregularity and the physiological response of the user. Displaying the navigation assistant output on the heads-up HMI can include generating one or more of an auditory and a visual user prompt requesting user feedback indicative of a desire to receive driving assistance, and providing, via the heads-up HMI, the navigation assistance to the user based on the user feedback. The system may generate one or more of an auditory and a visual user prompt requesting user feedback indicative of a desire to receive driving assistance, and provide, via the heads-up HMI, the navigation assistance to the user based on the user feedback.

At step 425, the method 400 may further include providing, via the processor, navigation assistance to the user. This step may include generating overtime data comprising historic driving patterns associated with the user, updating, via the processor, a breadcrumb database comprising a plurality of historic navigation maneuvers having turn-by-turn navigation data associated with a current trip, and compiling a breadcrumb map of the current trip based on the breadcrumb database. The navigation assistance to the user can include actions such as a telephonic communication to a family member offboard the vehicle, and/or displaying, via the heads-up HMI, turn-by-turn navigation.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A method for controlling a vehicle, comprising:
determining, via a processor, a driver state comprising a physiological response of a user operating the vehicle;
determining, via the processor, a vehicle route comprising a trip start position, path to a present position, and a trip destination;
identifying, via the processor, a navigation irregularity based on the vehicle route and the driver state;
displaying, via the processor, on a heads-up Human Machine Interface (HMI), a navigation assistant output based on the navigation irregularity and the physiological response of the user; and
providing, via the processor, navigation assistance to the user.

2. The method according to claim 1, further comprising:
determining, via the processor, a user identity based on user profile information.

3. The method according to claim 2, wherein determining the driver state further comprises:
determining the driver state based on the user identity.

4. The method according to claim 1, wherein determining the driver state further comprises:
determining the physiological response of the user by obtaining an ocular position of the user; and
determining a quantified stress level based on the ocular position of the user.

5. The method according to claim 1, wherein determining the driver state further comprises:
determining the physiological response of the user by sensing a user heart rate; and
determining a quantified stress level based on the user heart rate.

6. The method according to claim 1, wherein determining the driver state further comprises:
determining the physiological response of the user by obtaining a user respiration frequency; and
determining a quantified stress level based on the user respiration frequency.

7. The method according to claim 1, wherein determining the driver state further comprises:
determining the physiological response of the user by determining user blood pressure; and
determining a quantified stress level based on the user blood pressure.

8. The method according to claim 1, wherein identifying the navigation irregularity based on the vehicle route further comprises:
generating overtime data comprising historic driving patterns associated with the user;
updating, via the processor, a breadcrumb database comprising a plurality of historic navigation maneuvers having turn-by-turn navigation data associated with a current trip; and
compiling a breadcrumb map of the current trip based on the breadcrumb database.

9. The method according to claim 8, further comprising:
creating a breadcrumb map comparison of the breadcrumb map with the overtime data; and
identifying the navigation irregularity based on the breadcrumb map comparison and the driver state.

10. The method according to claim 1, wherein displaying the navigation assistant output on the heads-up HMI comprises generating one or more of an auditory and a visual user prompt requesting user feedback indicative of a desire to receive driving assistance; and
providing, via the heads-up HMI, the navigation assistance to the user based on the user feedback.

11. The method according to claim 1, wherein the navigation assistance comprises a telephonic communication to a family member offboard the vehicle.

12. The method according to claim 1, wherein the navigation assistance comprises:
displaying, via the heads-up HMI, turn-by-turn navigation.

13. A system disposed in a vehicle, comprising:
a heads-up Human Machine Interface (HMI),
a processor; and
a memory for storing executable instructions, the processor programmed to execute the instructions to:
determine a driver state comprising a physiological response of a user operating the vehicle;
determine a vehicle route comprising a trip start position, path to a present position, and a trip destination;
identify a navigation irregularity based on the vehicle route and the driver state;
display, on the heads-up HMI, a navigation assistant output based on the navigation irregularity and the physiological response of the user; and
provide navigation assistance to the user.

14. The system according to claim 13, wherein the processor is further programmed to execute the instructions to:
determine a user identity based on user profile information.

15. The system according to claim 14, wherein the processor is further programmed to determine the driver state based on the user identity.

16. The system according to claim 13, wherein the processor is further programmed to determine the driver state by executing the instructions to:
determine the physiological response of the user by sensing a user heart rate; and
determine a quantified stress level based on the user heart rate.

17. The system according to claim 13, wherein the processor is further programmed to determine the driver state by executing the instructions to:
determine the physiological response of the user by obtaining an ocular position of the user; and
determine a quantified stress level based on the ocular position of the user.

18. The system according to claim 13, wherein the processor is further programmed to identify the navigation irregularity based on the vehicle route by executing the instructions to:
generate overtime data comprising historic driving patterns associated with the user;
update a breadcrumb database comprising a plurality of historic navigation maneuvers having turn-by-turn navigation data associated with a current trip; and
compiling a breadcrumb map of the current trip based on the breadcrumb database.

19. The system according to claim 18, wherein the processor is further programmed to further execute the instructions to:
create a breadcrumb map comparison of the breadcrumb map with the overtime data; and
identify the navigation irregularity based on the breadcrumb map comparison and the driver state.

20. A non-transitory computer-readable storage medium in a vehicle computing device, the non-transitory computer-readable storage medium having instructions stored thereupon which, when executed by a processor, cause the processor to:
determine a driver state comprising a physiological response of a user operating a vehicle;
determine a vehicle route comprising a trip start position, path to a present position, and a trip destination;
identify a navigation irregularity based on the vehicle route and the driver state;
display, on a heads-up Human Machine Interface (HMI), a navigation assistant output based on the navigation irregularity and the physiological response of the user; and
provide navigation assistance to the user.
